(19) United States
(12) Patent Application Publication
(10) Pub. No.: US 2009/0170901 A1
(43) Pub. Date: Jul. 2, 2009

(54) Benzoyl Urea Derivatives

(76) Inventors: Istvan Borza, Budapest (HU); Gizella Bartané Szalai, Budapest (HU); Eva Bozo, Budapest (HU); Csilla Acsné Kiss, Budapest (HU); Csilla Horvath, Budapest (HU); Sandor Farkas, Budapest (HU); Jozsef Nagy, Budapest (HU); Sandor Kolok, Budapest (HU)

Correspondence Address: K.F. ROSS P.C., 5683 RIVERDALE AVENUE, SUITE 203 BOX 900, BRONX, NY, 10471-0900, US

(21) Appl. No.: 11/658,788

(22) PCT Filed: Jul. 21, 2005
(86) PCT No.: PCT/HU2005/000079

§ 371 Date: Jan. 25, 2007

(30) Foreign Application Priority Data
Jul. 29, 2004 (HU) P 0401524

(51) Int. Cl. A61K 31/445; C07D 211/60; A61P 25/00

(52) U.S. Cl. 514/330; 546/245

(57) ABSTRACT

The new benzoyl urea derivatives of formula (I) wherein the meaning of X and Y independently are hydrogen atom, hydroxy, benzyloxy, amino, nitro, C₁-C₄ alkylsulfonamido optionally substituted with a halogen atom or halogen atoms, C₁-C₄ alkanoylamido optionally substituted with a halogen atom or halogen atoms, C₁-C₄ alkoxy, aroyl-carbamoyl optionally substituted with halogen atom or C₁-C₄ alkyl or C₁-C₄ alkoxycarbonyl group, or the neighboring X and Y groups optionally form together with one or more identical or different additional hetero atom and —CH═ and/or —CH₂— groups an optionally substituted 4-7 membered homo- or heterocyclic ring, preferably morpholine, pyrrole, pyrrolidine, oxo- or thioxo-pyrrolidine, pyrazole, pyrazolidine, imidazole, imidazolidine, oxo- or thioxo-imidazole or imidazolidine, 1,4-oxazine, oxazole, oxazolidine, triazole, oxo- or thioxo-oxazolidine, or 3-oxo-1,4-oxazine ring, V and Z independently are hydrogen or halogen atom, cyano, C₁-C₄ alkyl, C₁-C₄ alkoxy, trifluoromethyl, hydroxy or optionally esterized carboxyl group, W is oxygen atom, as well as C₁-C₄ alkylene, C₂-C₄ alkenylene, aminocarbonyl, —NH—, —N(alkyl)-, —CH₂O—, —CH₂S—, —CH(OH)—, —OCH₂— group, wherein the meaning of alkyl is a C₁-C₄ alkyl group—, when the dotted bonds ( [symbol] ) represent simple C—C bonds then U is hydroxy group or hydrogen atom or when W is C₁-C₄ alkylene or C₂-C₄ alkenylene group, then one of the dotted bonds ( [symbol] ) can represent a further double C—C bond and in this case U means an electron pair, which participate in the double bond and optical antipodes, racemates and the salts thereof are highly effective and selective antagonists of NMDA receptor, and moreover most of the compounds are selective antagonist of NR2B subtype of NMDA receptor. Furthermore objects of the present invention are the pharmaceutical compositions containing new benzoyl urea derivatives of formula (I) or optical antipodes or racemates or the salts thereof as active ingredients and processes for producing these compounds and pharmaceutical compositions.

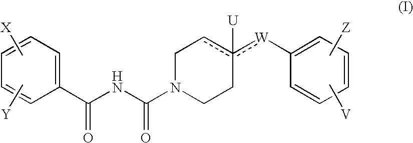

The invention relates to new benzoyl urea derivatives which are antagonists of NMDA receptor or are intermediates for preparing thereof.

### BACKGROUND OF THE INVENTION

N-methyl-D-aspartate (NMDA) receptors are ligand-gated cation-channels embedded in the cell membranes of neurons. Overactivation of NMDA receptors by glutamate, their natural ligand, can lead to calcium overload of cells. This triggers a cascade of intracellular events that alters the cell function and ultimately may lead to death of neurons [TINS, 10, 299-302 (1987)]. Antagonists of the NMDA receptors may be used for treating many disorders that are accompanied with excess release of glutamate, the main excitatory neurotransmitter in the central nervous system.

The NMDA receptors are heteromeric assemblies built up from at least 7 known subunit genes. The NR1 subunit is a necessary component of functional NMDA receptor channels. There are four genes encoding NR2 subunits (NR2A-D). Both spatial distributions in the CNS and the pharmacological sensitivity of NMDA receptors built up from various NR2 subunits are different. Recently, NR3A and NR3B have been reported. Particularly interesting of these is the NR2B subunit due to its restricted distribution (highest densities in the forebrain and substantia gelatinosa of the spinal cord). Compounds selective for this subtype are available and have been proved to be effective in animal models of stroke [Stroke, 28, 2244-2251 (1997)], traumatic brain injury [Brain Res., 792, 291-298 (1998)], Parkinson's disease [Exp. Neurol., 163, 239-243 (2000)], neuropathic and inflammatory pain (Neuropharmacology, 38, 611-623 (1999)]. Moreover, NR2B subtype selective antagonists of NMDA receptors are expected to possess little or no untoward side effects that are typically caused by the non-selective antagonists of NMDA receptors, namely psychotornimetic effects such as dizziness, headache, hallucinations, dysphoria and disturbances of cognitive and motor function.

NR2B subtype selective NMDA antagonism can be achieved with compounds that specifically bind to, and act on, an allosteric modulatory site of the NR2B subunit containing receptors. This binding site can be characterized by displacement (binding) studies with specific radioligands, such as [¹²⁵I]-ifenprodil [J. Neurochem., 61, 120-126 (1993)] or [³H]-Ro 25, 6981 [J. Neurochem., 70, 2147-2155 (1998)]. Since ifenprodil was the first, though not sufficiently specific, known ligand of this receptor, it has also been termed ifenprodil binding site.

Close structure analogs of the benzoyl urea derivatives of formula (I) are unknown from the literature.

### SUMMARY OF THE INVENTION

Surprisingly it was found that the new benzoyl urea derivatives of formula (I) of the present invention are functional antagonists of NR2B subunit containing NMDA receptors, while they are ineffective on NR2A subunit containing NMDA receptors. Therefore, they are believed to be NR2B subtype specific NMDA antagonists. Some compounds proved to be effective in vivo in mouse pain model after oral administration.

### DETAILED DESCRIPTION OF THE INVENTION

The present invention relates therefore first to new benzoyl urea derivatives of formula (I)

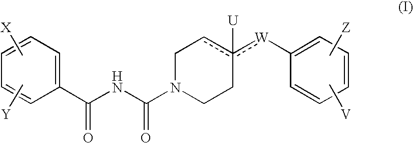

wherein the meaning of
X and Y independently are hydrogen atom, hydroxy, benzyloxy, amino, nitro, C₁-C₄ alkylsulfonamido optionally substituted with a halogen atom or halogen atoms, C₁-C₄ alkanoylamido optionally substituted with a halogen atom or halogen atoms, C₁-C₄ alkoxy, aroyl-carbamoyl optionally substituted with halogen atom or C₁-C₄ alkyl or C₁-C₄ alkoxycarbonyl group, or
the neighboring X and Y groups optionally form together with one or more identical or different additional hetero atom and —CH═ and/or —CH₂— groups an optionally substituted 4-7 membered homo- or heterocyclic ring, preferably morpholine, pyrrole, pyrrolidine, oxo- or thioxo-pyrrolidine, pyrazole, pyrazolidine, imidazole, imidazolidine, oxo- or thioxo-imidazole or imidazolidine, 1,4-oxazine, oxazole, oxazolidine, triazole, oxo- or thioxo-oxazolidine, or 3-oxo-1,4-oxazine ring,
Y and Z independently are hydrogen or halogen atom, cyano, C₁-C₄ alkyl, C₁-C₄ alkoxy, trifluoromethyl, hydroxy or optionally esterized carboxyl group,
W is oxygen atom, as well as C₁-C₄ alkylene, C₂-C₄ alkenylene, aminocarbonyl, —NH—, —N(alkyl)-, —CH₂O—, —CH₂S—, —CH(OH)—, —OCH₂— group, —wherein the meaning of alkyl is a C₁-C₄ alkyl group—,
when the dotted bonds [symbol] represent simple C—C bonds then U is hydroxy group or hydrogen atom or
when W is C₁-C₄ alkylene or C₂-C₄ alkenylene group, then one of the dotted bonds [symbol] can represent a further double C—C bond and in this case U means an electron pair, which participate in the double bond
   and optical antipodes, racemates and the salts thereof.

Furthermore objects of the present invention are the pharmaceutical compositions containing, new benzoyl urea derivatives of formula (I) or optical antipodes or racemates or the salts thereof as active ingredients.

Further objects of the invention are the processes for producing new benzoyl urea derivatives of formula (I), and the pharmaceutical manufacture of medicaments containing these compounds, as well as the process of treatments with these compounds, which means administering to a mammal to be treated—including human—effective amount/amounts of new benzoyl urea derivatives of formula (I) of the present invention as such or as medicament.

The new benzoyl urea derivatives of formula (I) of the present invention are highly effective and selective antagonists of NMDA receptor, and moreover most of the compounds are selective antagonist of NR2B subtype of NMDA receptor.

According to the invention the new benzoyl urea derivatives of formula (I) can be synthesized as follows:
a.) by reacting a substituted benzoyl isocyanate of formula (II) preferably synthesized in situ

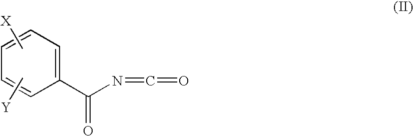

wherein the meaning of X and Y are as described before for the formula (I)—with an amine of formula (III)

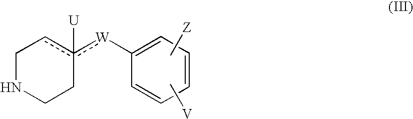

wherein the meaning of V, W, Z, the dotted bonds [symbol] and U are as described before for the formula (I)—in a solvent, or
b.) by coupling a substituted benzamide of formula (V)

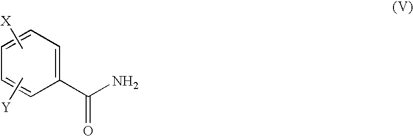

where the meaning of X is hydroxy group and Y is as described before for the formula (I)—onto a resin using triphenyl phosphine and diethyl azodicarboxylate, then

reacting the obtained benzamide coupled to resin with oxalyl chloride and the so formed benzoyl isocyanate is further reacted with an amine of formula (III)

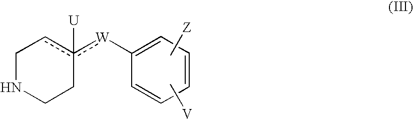

wherein the meaning of V, W, Z, the dotted bonds [symbol] and U are as described before for the formula (I)—in the presence of a trialkyl amine,

and finally splitting off the obtained benzoyl urea derivatives of formula (I)—wherein the meaning of X, Y, V, W, Z, the dotted bonds [symbol] and U are as described before for the formula (I)—from the resin.

Then the benzoyl urea derivatives of formula (I) —wherein the meaning of X, Y, V, W, Z, the dotted bonds [symbol] and U are as described before for the formula (I)—obtained in the process a.) or b.) are optionally transformed into another compound of formula (I) by introducing new substituents and/or modifying or removing the existing ones, and/or by salt formation and/or by liberating the compound from salts, and/or by resolving the obtained racemates using optically active acids or bases by known methods.

The compounds of this, invention are readily prepared in process a.) by reacting the appropriate benzoyl isocyanate with an appropriate amine in a reaction-inert solvent at a temperature of from about 0° C. to about 20° C. Representative solvents for these reactions are methylene chloride, ethylene dichloride, tetrahydrofuran, dioxane, diethyl ether, dimethyl ether of ethylene glycol, benzene, toluene and xylene.

The requisite isocyanates are conveniently prepared by reacting the corresponding amide with oxalyl chloride (U.S. Pat. No. 4,163,784) or by condensation of aroyl chlorides with sodium cyanate [Tetrahedron, 44, 6079-6086. (1988)]. The amide reactants used to prepare the isocyanate reactants are prepared by amidation of the corresponding acid chlorides according to well known procedures. The acid chlorides are prepared by reaction of the appropriate carboxylic acid with thionyl chloride, the latter generally serving as reactant and solvent. The isocyanate need not to be isolated from the reaction mixture. The isocyanate and amine are generally used in equimolar ratios. A proper amine of formula (III) is added as base or as a salt formed with inorganic acid to the so obtained solution or suspension in the presence of a base, for example trietylamine, needed for the liberation of the amine. The necessary reaction time is 0-1 h. The work-up of the reaction mixture can be carried out by different methods.

When the isocyanate reactants are prepared from the corresponding amides, at the end of the addition of the amine the reaction mixture is washed with water and concentrated. The residue is crystallized or purified by column chromatography. When the reaction mixture is a suspension, the precipitate is filtered off, washed with water and recrystallized from a proper solvent to give the pure product. When the isocyanate reactants are prepared by condensation, of aroyl chlorides with sodium cyanate, at the end of the addition the reaction mixture is concentrated and the residue is crystallized from a proper solvent to give the pure product. If the crystallization does not lead to the pure product, then column chromatography can be used for the purification of it. The column chromatography is carried out on normal phase using Kieselgel 60 as adsorbent and different solvent systems, e.g. toluene/methanol, chloroform/methanol or toluene/acetone, as eluents. The structure of the products are determined by IR, NMR and mass spectrometry.

In the solid phase synthesis as described in process b.) you can preferably use resins which have hydroxy-methyl (—CH₂—OH) groups as active moiety. The most preferably used resin is the so called Wang resin from Novabiochem.

The obtained benzoyl urea derivatives of formula (I) —independently from the method of preparation—optionally can be transformed into an other compound of formula (I) by introducing further substituents and/or modifying and/or removing the existing ones, and/or formation of salts with acids and/or liberating the carboxylic acid amide derivative of formula (I) from the obtained acid addition salts by treatment with a base and/or the free carboxylic acid amide derivative of formula (I) can be transformed into a salt by treatment with a base.

For example cleaving the methyl and benzyl groups from methoxy and benzyloxy groups, which stands for U, V and Z, leads to phenol derivatives. The removal of the benzyl group can be carried out for example with catalytic hydrogenation or with hydrogen bromide in acetic acid solution, the cleavage of methyl group can be carried out with boron tribromide in dichloromethane solution.

Free hydroxy groups can be esterified by acid anhydrides or acid halogenides in the presence of a base.

The benzoyl isocyanate of formula (II) can be synthesized by different known methods from corresponding amides or aroyl chlorides. The syntheses of some commercially not available amides or aroyl chlorides are described in the Examples.

### Experimental Protocols

### Expression of Recombinant NMDA Receptors

To prove NR2B selectivity of our compounds, we tested them on cell lines stably expressing recombinant NMDA receptors with subunit compositions of NR1/NR2A or NR1/NR2B. cDNAs of human NR1-3 and NR2A or rat NR1a and NR2B subunits subcloned into inducible mammalian expression vectors were introduced into HEK 293 cells lacking NMDA receptors using a cationic lipid-mediated transfection method [Biotechniques, 1997 May; 22(5), 982-7. (1997); Neurochemistry International, 43, 19-29. (2003)]. Resistance to neomycin and hygromycin was used to screen for clones possessing both vectors and monoclonal cell lines were established from the clones producing the highest response to NMDA exposure. Compounds were tested for their inhibitory action on NMDA evoked cytosolic calcium elevations in fluorescent calcium measurements. Studies were performed 48-72 h after addition of the inducing agent. Ketamine (500 μM) was also present during the induction in order to prevent cytotoxicity.

Assessing the Functional NMDA Antagonist Potency of Compounds on HEK293 cells expressing Recombinant NMDA Receptors Based on Measuring the Intracellular Calcium Concentration Using a Fluorimeter Plate Reader

Since NMDA receptors are known to be permeable to calcium ions upon excitation, the extent of NMDA receptor activation, and its inhibition by functional antagonists can be characterised by measuring the rise in the intracellular calcium concentration following agonist (NMDA) application onto the cells. Since there is very high sequence homology between rat and human NMDA receptors (99, 95, 97% for NR1, NR2A, and NR2B subunits, respectively), it is believed that there is little, if any, difference in their pharmacological sensitivity. Hence, results obtained with (cloned or native) rat NMDA receptors may be well extrapolated to the human ones.

The intracellular calcium measurements are carried out on HEK293 cells expressing NR1a and NR2B or NR2A NMDA receptor subunits. Cells are plated onto standard 96-well microplates and the cultures are maintained in an atmosphere of 95% air-5% CO₂ at 37° C. until testing.

The cells are loaded with a fluorescent Ca²⁺-sensitive dye, Fluo-4/AM (2-2.5 μM) prior to testing. Loading is stopped by washing twice with the solution used also during the measurement (140 mM NaCl 5 mM KCl, 2 mM CaCl₂, 5 mM HEPES [4-(2-hydroxyethyl)-1-piperazineethane-sulfonic acid], 5 mM HEPES—Na, 20 mM glucose, 10 μM glycine, pH=7.4). Then the test compound dissolved in the above solution (90 μl/well) is added. Intracellular calcium measurements are carried out with a plate reader fluorimeter. A rise in Fluo-4-fluorescence that reflects the intracellular calcium concentration is induced by application of 200 μM NMDA. Inhibitory potency of the test compound is assessed by measuring the reduction in the calcium elevation in the presence of different concentrations of the compound.

Inhibitory potency of a compound at a single concentration point is expressed as percent inhibition of the control NMDA response. For NR1a/NR2B expressing cells concentration-inhibition curves are produced. Sigmoidal concentration-inhibition curves are fitted over the data and IC₅₀ values are defined as the concentration that produces half of the maximal inhibition that could be achieved with the compound. Mean IC₅₀ values are derived from at least three independent experiments. For NR1-3/NR2A expressing cells antagonism of NMDA induced rise in intracellular calcium concentration by compounds of the present invention and reference compounds was tested at 10 and 15 microM concentration, respectively.

### The Biological Activity of the Compounds

IC₅₀ values determined in NR1a/NR2B transfected cells and percentage inhibition at 15 μM concentration in NR1-3/NR2A transfected cells are listed in Table 1 for selected examples of compounds of this invention. For comparison, data for the most potent known reference compounds were also determined and are given in Table 2.

The compounds of this invention exhibit IC₅₀ values of less than 15 μM in the functional NMDA antagonism test in NR1/NR2B transfected cells, and are inactive at this concentration on NR1-3/NR2A transfected cells. Thus the compounds and pharmaceutical compositions of this invention are NR2B subtype specific NMDA antagonists. Some of the compounds have superior potency-compared to the known reference compounds (see Table 1).

TABLE 1
NMDA antagonist activity of compounds measured
by fluorimetric method on cells expressing
NR1a/NR2B or NR1-3/NR2A subunits

| Compound of | NR1a/NR2B | | NR1-3/NR2A | |
|---|---|---|---|---|
| Example | IC₅₀ [nM] | n | % inhibition at 15 uM | n |
| 1 | 28.0 | 2 | 2.6 | 1 |
| 2 | 7.6 | 2 | −5.7 | 1 |
| 3 | 6.2 | 2 | 8.7 | 1 |
| 5 | 19.3 | 2 | 4.9 | 1 |
| 4 | 5.3 | 2 | 1.8 | 1 |
| 7 | 59.6 | 2 | −6.7 | 1 |
| 6 | 15.0 | 2 | −2.9 | 1 |
| 26 | 58.9 | 2 | −0.9 | 1 |
| 27 | 14.3 | 2 | 11.4 | 1 |
| 28 | 59.1 | 2 | −2.9 | 1 |
| 29 | 8.3 | 2 | 19.7 | 1 |

TABLE 2
NMDA antagonist activity of reference compounds
measured by fluorimetric method on cells expressing
NR1a/NR2B or NR1-3/NR2A subunits

| Code of | NR1a/NR2B | | NR1-3/NR2A | |
|---|---|---|---|---|
| reference compound | IC₅₀ [nM] | n | % inhibition at 10 uM | n |
| CI-1041 | 8.4 | 4 | 21.0 | 1 |
| Co-101244 | 4.8 | 3 | −8.7 | 1 |
| EMD 95885 | 48 | 1 | 0.1 | 1 |
| CP 101,606 | 30 | 3 | 2.5 | 1 |
| Ro 25.6981 | 57 | 4 | 1.0 | 1 |
| ifenprodil | 459 | 5 | −2.7 | 1 |
| MK-801 | 43 | 3 | IC₅₀ = 386 nM | 2 |

The reference compounds are as follows:

CI-1041: 6-{2-[4-(4-fluoro-benzyl)-piperidin-1-yl]-ethanesulfinyl}-3H-benzooxazol-2-one
Co 101244: 1-[2-(4-hydroxyphenoxy)ethyl]-4-hydroxy-4-(4-methylbenzyl)piperidine
EMD 95885: 6-[3-(4-fluorobenzyl)piperidine-1-yl]propionyl]-2,3-dihydro-benzoxazol-2-on
CP-101,606: (1S,2S)-1-(4-hydroxyphenyl)-2-(4-hydroxy-4-phenylpiperidine-1-yl)-1-propanol
Ro 256981: R—(R*,S*)-1-(4-hydroxyphenyl)-2-methyl-3-[4-(phenylmethyl)piperidin-1-yl]-1-propanol.
Ifenprodil: erythro-2-(4-benzylpiperidino)-1-(4-hydroxyphenyl)-1-propanol
MK-801: (+)-5-Methyl-10,11-dihydro-5H-dibenzo[a,d]cyclohepten-5,10-imine
Mouse formalin Test for Measurement of In Vivo Efficacy

Injection of diluted formalin into the hind paw of rats or mice is known to elicit a biphasic pain-related behavior measured as time spent by licking/biting of the injured paw. The second phase is generally defined as pain related events detected in the 15-60 min. time interval after formalin injection. It is known that NMDA receptors are involved in the second phase of response to formalin injection and this behavioral response is sensitive to blockade of NMDA receptors [Dickenson, A. and Besson J.-M. (Editors): Chapter 1, pp. 6-7: Animal models of Analgesia; and Chapter 8, pp. 180-183: Mechanism of Central Hypersensitivity: Excitatory Amino Acid Mechanisms and Their Control—In Pharmacology of Pain. Springer-Verlag (Berlin)> 1997.] Therefore, we used the second phase of formalin test to characterize the efficacy of compounds in vivo. Inhibition of the second phase of response is considered to indicate an analgesic effect against chemically-induced persistent pain [Hunker, S., et al.: Formalin Test in Mice, a Useful Technique for Evaluating Mild Analgesics, Journal of Neuroscience Methods, 14 (1985)69-76.]

Male albino Charles River NMRI mice (20-25 g) were used. Prior to the experiment any solid food was withdrawn for approx. 16 hours but the animals had free access to 20% glucose solution. The animals were allowed 1 hour acclimatization period in a glass cylinder (cc. 15 cm in diameter), then moved to an identical cylinder with a mirror placed behind to facilitate observation. The test substances were suspended in 5% tween-80 (10 ml per kg body weight). and administered orally by gavage 15 min before the formalin injection (20 μl of 1% formalin in 0.9% saline injected subcutaneously into the dorsal surface of the right hindpaw). The time spent by licking and biting of the injected paw was measured from 20 to 25 min. after the formalin injection. For the determination of ED₅₀ value, various doses (at least five) of the test substances were given to groups of 5 mice and the results expressed as % inhibition time spent by licking relative to a vehicle control group observed on the same day. ED₅₀ values (i.e. the dose yielding 50% inhibition) were calculated by Boltzman's sigmoidal curve fitting. ED₅₀ values are listed in Table 3 for selected examples of compounds of this invention and reference compounds.

TABLE 3
ED₅₀ values of selected compounds

| | ED₅₀ (mg/kg p.o. |
|---|---|
| | or as indicated) |
| Compound of Example | |
| 1 | 1.6 |
| 5 | 27 |
| ID code of compounds | |
| CI-1041 | 2.4 |
| Co-101244 | >20* |
| | (5.9 mg/kg i.p.) |
| EMD 95885 | 3.7 |
| CP-101,606 | >20* |
| Ro-256981 | >20* |
| | (5.1 mg/kg i.p.) |

*ED₅₀ value was not determined if the inhibition was less than 50% at the dose of 20 mg/kg, p.o.

Disorders which may be beneficially treated with NMDA antagonists acting at NR2B site, as reviewed recently by Loftis [Pharmacology & Therapeutics, 97, 55-85 (2003)] include schizophrenia, Parkinson's disease, Huntington's disease, excitotoxicity evoked by hypoxia and ischemia, seizure disorders, drug abuse, and pain, especially neuropathic, inflammatory and visceral pain of any origin [Eur. J. Pharmacol., 429, 71-78 (2001)].

Due to their reduced side effect liability compared to non-selective NMDA antagonists, NR2B selective antagonists may have utility in diseases where NMDA antagonist may be effective, such as amyotrophic lateral sclerosis [Neurol. Res., 21, 309-12 (1999)], withdrawal syndromes of e.g. alcohol, opioids or cocaine [Drug and Alcohol Depend., 59, 1-15 (2000)], muscular spasm [Neurosci. Lett., 73, 143-148 (1987)], dementia of various origins [Expert Opin. Investig. Drugs, 9, 1397-406 (2000)], anxiety, depression, migraine, hypoglycemia, degenerative-disorders of the retina (e.g. CMV retinitis), glaucoma, asthma, tinnitus, hearing loss [Drug News Perspect, 11, 523-569 (1998) and WO. 00/00197 international patent application].

Accordingly, effective amounts of the compounds of the invention may be beneficially used for the treatment of traumatic injury of brain or spinal cord, tolerance and/or dependence to opioid treatment of pain, development of tolerance, decrease of abuse potential and withdrawal syndromes of drugs of abuse e.g. alcohol, opioids or cocaine, ischemic CNS disorders, chronic neurodegenerative disorders, such as e.g. Alzheimer's disease, Parkinson's disease, Huntington's disease, pain and chronic pain states, such as e.g. neuropathic pain.

The compounds of the invention as well as their pharmaceutically acceptable salts can be used as such or suitably in the form of pharmaceutical compositions. These compositions (drugs) can be in solid, liquid or semiliquid form and pharmaceutical adjuvant and auxiliary materials can be added, which are commonly used in practice, such as carriers, excipients, diluents, stabilizers, wetting or emulsifying agents, pH- and osmotic pressure-influencing, flavoring or aromatizing, as well as formulation-promoting or formulation-providing additives.

The dosage required to exert the therapeutical effect can vary within wide limits and will be fitted to the individual requirements in each of the particular cases, depending on the stage of the disease, the condition and the bodyweight of the patient to be treated, as well as the sensitivity of the patient against the active ingredient, route of administration and number of daily treatments. The actual dose of the active ingredient to be used can safely be determined by the attending physician skilled in the art in the knowledge of the patient to be treated.

The pharmaceutical compositions containing the active ingredient according to the present invention usually contain 0.01 to 100 mg of active ingredient in a single dosage unit. It is, of course possible that the amount of the active ingredient in some compositions exceeds the upper, or lower limits defined above.

The solid forms of the pharmaceutical compositions can be for example tablets, dragées, capsules, pills or lyophilized powder ampoules useful for the preparation of injections. Liquid compositions are the injectable and infusible compositions, fluid medicines, packing fluids and drops. Semiliquid compositions can be ointments, balsams, creams, shaking mixtures and suppositories.

For the sake of a simple administration it is suitable if the pharmaceutical compositions comprise dosage units containing the amount of the active ingredient to be administered once, or a few multiples or a half, third or fourth part thereof. Such dosage units are e.g. tablets, which can be powdered with grooves promoting the halving or quartering of the tablet in order to exactly administer the required amount of the active ingredient.

Tablets can be coated with an acid-soluble layer in order to assure the release of the active ingredient content after leaving the stomach. Such tablets are enteric-coated. A similar effect can be achieved also by encapsulating the active ingredient.

The pharmaceutical compositions for oral administration can contain e.g. lactose or starch as excipients, sodium carboxymethylcellulose, methylcellulose, polyvinyl pyrrolidine or starch paste as binders or granulating agents. Potato starch or microcrystalline cellulose is added as disintegration agents, but ultraamylopectin or formaldehyde casein can also be used. Talcum, colloidal silicic acid, stearin, calcium or magnesium stearate can be used as antiadhesive and lubricants.

The tablet can be manufactured for example by wet granulation, followed by pressing. The mixed active ingredients and excipients, as well as in given case part of the disintegrants are granulated with an aqueous, alcoholic or aqueous alcoholic, solution of the binders in an appropriate equipment, then the granulate is dried. The other disintegrants, lubricants and antiadhesive agents are added to the dried granulate, and the mixture is pressed to a tablet. In given case the tablets are made with halving groove to ease the administration.

The tablets can be made directly from the mixture of the active ingredient and the proper auxiliaries by pressing. In given case, the tablets can be coated by using additives commonly used in the pharmaceutical practice, for example stabilizers, flavoring, coloring agents, such as sugar, cellulose derivatives (methyl- or ethylcellulose, sodium carboxymethylcellulose, etc), polyvinyl pyrrolidone, calcium phosphate, calcium carbonate, food coloring agents, food laces, aroma agents, iron oxide pigments, etc. In the case of capsules the mixture of the active ingredient and the auxiliaries is filled into capsules.

Liquid oral compositions, for example suspensions, syrups, elixirs can be made by using water, glycols, oils, alcohols, coloring and flavoring agents.

For rectal administration the composition is formulated in suppositories or clysters. The suppository can contain beside the active ingredient a carrier, so called adeps pro suppository. Carriers can be vegetable oils, such as hydrogenated vegetable oils, triglycerides of C₁₂-C₁₈ fatty acids (preferably the carriers under the trade name Witepsol). The active ingredient is homogeneously mixed with the melted adeps pro suppository and the suppositories are moulded.

For parenteral administration the composition is formulated as injection solution. For manufacturing the injection solution the active ingredients are dissolved in distilled water and/or in different organic solvents, such as glycolethers, in given case in the presence of solubilizers, for example polioxyethylensorbitane-monolaurate, -monooleate, or monostearate (Tween 20, Tween 60, Tween 80). The injection solution can also contain different auxiliaries, such as conserving agents, for example ethylendiamine tetraacetate, as well as pH adjusting agents and buffers and in given case local anaesthetic, e.g. lidocain. The injection solution containing the active ingredient of the invention is filtered before it is filled into ampoules, and it is sterilized after filling.

If the active ingredient is hygroscopic, then it can be stabilized by liophylization.

### Characterization Method in the Case of Solid Phase Synthesis

Compounds of the present invention were characterized by high performance liquid chromatography coupled to mass selective detector (LC/MS) using HP 1100 Binary Gradient chromatography system with Microplate Sampler (Agilent, Waldbronn), controlled by ChemStation software. HP diode array detector was used to acquire UV spectra at 225 and 240 nm. All experiments were performed using HP MSD (Agilent, Waldbronn) single quadruple spectrometer equipped with an electrospray ionization source to determine the structure.

The synthesized products were dissolved in 1 ml of DMSO (Aldrich, Germany). 100 μl of each solution was diluted with DMSO to 1000 μl volume. Analytical chromatographic experiments were performed on Discovery RP C-16 Amide, 5 cm×4.6 mm×5 μm column from Supelco (Bellefonte, Pa.) with a flow rate of 1 ml/minute for qualification. The obtained compounds were characterized by their k′ value (purity, capacity factor), k′ factors are evaluated by the following formula:

k′=(t_{R}−t₀)/t₀

where k′=capacity factor, t_{R}=retention time and t₀=eluent retention time.

The A eluent was trifluoroacetic acid (TFA) (Sigma, Germany) containing 0.1% water, the B eluent was 95% acetonitrile (Merck, Germany) containing 0.1% TFA and 5% A eluent. Gradient elution was used, starting with 100% A eluent and processing to 100% B eluent over a period of 5 minutes.

The following examples illustrate the invention without the intention of limitation anyway.

### Method A.

### Example 1

### 4-Benzyl-piperidine-1-carboxylic Acid 4-hydroxy-benzoylamide

### 1a) 4-Benzyl-piperidine-1-carboxylic Acid 4-benzyloxy-benzoylamide

Under argon, to a stirred solution of 1.62 g (6.57 mmol) of 4-benzyloxy-benzoyl chloride [Liebigs Ann. 10, 2169-2176. (1997)] and 0.57 g (8.7 mmol) of sodium cyanate in 10 ml of acetonitrile and 10 ml of benzene 36 μl (0.3 mmol) of tin(IV) chloride is added. The reaction mixture is refluxed for 3 h, cooled to 20° C., then 1.17 g (6.57 mmol) of 4-benzyl-piperidine (Aldrich) is added drop wise at 20° C. The reaction mixture is stirred at 20° C. for 1 h, concentrated, the residue is treated with methanol and the crystals are filtered to yield 1.07 g (38%) of the title compound. Mp.: 155-156° C.

### 1b) 4-Benzyl-piperidine-1-carboxylic Acid 4-hydroxy-benzoylamide

A mixture of 1.07 g (2.5 mmol) of 4-benzyl-piperidine-1-carboxylic acid 4-benzyloxy-benzoylamide, 20 ml of tetrahydrofuran, 20 ml of methanol and 0.5 g of 10% Pd/C catalyst is hydrogenated for 2 h. The catalyst is filtered off, washed with tetrahydrofuran and the filtrate is concentrated. The residue is purified by column chromatography using Kieselgel 60 as adsorbent (Merck) and toluene:methanol=4:1 as eluent to yield 0.48 g (56.7%) of the title compound. Mp.: 95° C. (diisopropyl ether).

### Example 2

### 4-(4-Methoxy-benzyl)-piperidine-1-carboxylic Acid 4-hydroxy-benzoylamide

### 2a) 4-(4-Methoxy-benzyl)-piperidine-1-carboxylic Acid 4-benzyloxy-benzoylamide

The title compound is prepared from 4-benzyloxy-benzoyl chloride and (4-methoxy-benzyl)-piperidine [U.S. Pat. No. 3,632,767 (1972)] according to the method described in Example 1a.

### 2b) 4-(4-Methoxy-benzyl)-piperidine-1-carboxylic Acid 4-hydroxy-benzoylamide

The title compound is prepared from 4-(4-methoxy-benzyl)-piperidine-1-carboxylic acid 4-benzyloxy-benzoylamide according to the method described in Example 1b. Mp.: 190° C.

### Example 3

### 4-(4-Methyl-benzyl)-piperidine-1-carboxylic Acid 4-hydroxy-benzoylamide

### 3a) 4(4-Methyl-benzyl)-piperidine-1-carboxylic Acid 4-benzyloxy-benzoylamide

A mixture of 2.1 g (10 mmol) of 4-methanesulfonylamino-benzamide [J. Org. Chem., 66, 8299. (2001)], 1.3 ml (15 mmol) of oxalyl chloride (Aldrich) and 10 ml of 1,2-dichloroethane is refluxed for 3 hours and then cooled to 5° C. 2.3 ml (12 mmol) of 4-(4-methyl-benzyl)-piperidine [J. Org. Chem. 64, 3763. (1999)] in 5 ml of 1,2-dichloroethane is added drop wise below 10° C., and the reaction mixture is stirred at room temperature for 5 hours. Then it is poured into 25 ml of water, the resultant crystals were collected by filtration and washed with water to yield 2.36 g (55%) of the title compound. Mp.: 204-208° C. (1,2-dichloroethane-water).

### Example 7

### 4-Benzyl-piperidine-1-carboxylic Acid (2-oxo-2,3-dihydro-benzooxazole-6-carbonyl)-amide

### 7a) 2-Oxo-2,3-dihydro-benzooxazole-6-carboxylic Acid Amide

To a stirred solution of 0.37 g (2.06 mmol) of 2-oxo-2,3-dihydro-benzooxazole-6-carboxylic acid [Eur. J. Med. Chem. Chim. Ther., 9, 491-492. (1974)], 13 ml of 1,4-dioxane and 0.1 ml of dimethylformamide 1.35 ml (18 mmol) of thionyl chloride is added drop wise below 10° C., and the reaction mixture is stirred at room temperature for 24 h. Then 10 ml of 25% ammonium hydroxide solution is added drop wise to the mixture. The reaction mixture is concentrated and the residue is purified by column chromatography using Kieselgel 60 as adsorbent (Merck) and chloroform:methanol=3:1 as eluent to yield 0.13 g (35.3%) of the title compound. Mp.: 296° C. (2-propanol).

### 7b) 4-Benzyl-piperidine-1-carboxylic Acid (2-oxo-2,3-dihydro-benzooxazole-6-carbonyl)-amide

The title compound is prepared from 2-oxo-2,3-dihydro-benzooxazole-6-carboxylic acid amide according to the method described in Example 6. Mp.: 174° C.

### Example 8

### 4-(4-tert-Butyl-benzyl)-piperidine-1-carboxylic Acid 4-hydroxy-benzoylamide

### 8a) 4-(4-tert-Butyl-benzyl)-piperidine-1-carboxylic Acid 4-benzyloxy-benzoylamide

The title compound is prepared from 4-benzyloxy-benzoyl chloride and 4-(4-tert-butyl-benzyl)-piperidine [J. Org. Chem., 64, 3763. (1999)] according to the method described in Example 1a.

### 8b) 4-(4-tert-Butyl-benzyl-piperidine-1-carboxylic Acid 4-hydroxy-benzoylamide

The title compound is prepared from 4-(4-tert-butyl-benzyl)-piperidine-1-carboxylic acid 4-benzyloxy-benzoylamide according to the method described in Example 1b. Mp.: 101° C.

### Example 9

### 4-(4-Chloro-phenoxy)-piperidine-1-carboxylic Acid 4-hydroxy-benzoylamide

### 9a) 4-(4-Chloro-phenoxy)-piperidin-1-carboxylic Acid tert-butyl ester

Under argon, to a stirred solution of 10.0 g (49.7 mmol) of 4-hydroxy-piperidin-1-carboxylic acid tert-butyl ester [Bioorg. Med. Chem. Lett., 10, 2815. (2000)] in 80 ml of dimethylformamide 3.0 g (60% , 75 mmol) of sodium hydride is added. The reaction mixture is stirred at 40° C. for 1 h, then 5.3 ml (49.7 mmol) of 1-chloro-4-fluoro-benzene (Aldrich) in 20 ml of dimethylformamide is added drop wise at 20° C. The reaction mixture is stirred at 80° C. for 4 h, cooled to 20° C., 1 ml of ethanol is added drop wise, poured into 100 ml of water and extracted with ethyl acetate. The organic layer is dried over sodium sulfate and concentrated. The residue is purified by column chromatography using Kieselgel 60 (Merck) as adsorbent and ethyl acetate as eluent to yield 11.07 g (75.5%) of the title compound. Mp.: oil

### 9b) 4-(4-Chloro-phenoxy)-piperidine Hydrochloride

To a solution of 150 ml of 2.5 M hydrochloric acid in ethyl acetate 11.07 g (37.5 mmol) of 4-(4-chloro-phenoxy)-piperidin-1-carboxylic acid tert-butyl ester is added. The reaction mixture, is stirred at 20° C. for 3 h, then concentrated to 50 ml. The precipitated crystals are filtered off, washed with ethyl acetate to yield 7.0 g (75.2%) of the title compound. Mp.: 194-196° C.

### 9c) 4-(4-Chloro-phenoxy)-piperidine-1-carboxylic Acid 4-benzyloxy-benzoylamide

The title compound is prepared from 4-benzyloxy-benzoyl chloride and 4-(4-chloro-phenoxy)-piperidine according to the method described in Example 1a.

### 9d) 4-(4-Chloro-phenoxy)-piperidine-1-carboxylic Acid 4-hydroxy-benzoylamide

The title compound is prepared from 4-(4-chloro-phenoxy)-piperidine-1-carboxylic acid 4-benzyloxy-benzoylamide according to the method described in Example 4b. Mp.: 189° C.

### Example 10

### 4-Phenoxymethyl-piperidine-1-carboxylic Acid 4-hydroxy-benzoylamide

### 10a) 4-Phenoxymethyl-piperidine-1-carboxylic Acid 4-benzyloxy-benzoylamide

The title compound is prepared from 4-benzyloxy-benzoyl chloride and 4-phenoxy-methyl-piperidine [DE 254 999 (1977)] according to the method described in Example 1a.

### 10b) 4-Phenoxymethyl-piperidine-1-carboxylic Acid 4-hydroxy-benzoylamide

The title compound is prepared from 4-phenoxymethyl-piperidine-1-carboxylic acid 4-benzyloxy-benzoylamide according to the method described in Example 1b. Mp.: 207° C.

### Example 11

4-(2,4-Difluoro-benzyl)-piperidine-1-carboxylic Acid 4-hydroxy-benzoylamide

### 11a) 4-(2,4-Difluoro-benzylidene)piperidin-1-carboxylic Acid tert-butyl Ester

Under argon, to a stirred solution of 4.1 g (20.6 mmol) of N-(tert-butoxycarbonyl)-4-piperidone and 5.42 g (20.5 mmol) of (2,4-difluoro-benzyl)-phosphoric acid diethyl ester [Eur. J. Med. Chim. Ther., 27, 845. (1992)] in 50 ml of dimethylformamide 1.3 g (60%, 32.5 mmol) of sodium hydride is added at 0° C. The reaction mixture is stirred at 20° C. for 4 h, 1 ml of ethanol is added drop wise, poured into 100 ml of water and extracted with diethyl ether. The organic layer is dried over sodium sulfate and concentrated. The crude product is used in the next step.

Yield: 5.1 g (80.7%). Mp.: oil.

### 11b) 4-(2,4-Difluoro-benzyl)-piperidin-1-carboxylic Acid tert-butyl Ester

A mixture of 5.1 g (14.69 mmol) of 4-(2,4-difluoro-benzylidene-piperidin-1-carboxylic acid tert-butyl ester, 200 ml of ethanol and 0.5 g of 10% Pd/C catalyst is hydrogenated. After completion of the reaction, the catalyst is filtered off, washed with tetrahydrofuran and the filtrate is concentrated. The crude product is used in the next step. Yield: 5.2 g (100%). Mp.: oil.

### 11c) 4-(2,4-Difluoro-benzyl)-piperidine

The title compound is prepared from 4-(2,4-difluoro-benzyl)-piperidin-1-carboxylic acid tert-butyl ester according to the method described in Example 9b. Mp.: 191° C. (ethyl acetate-diethyl ether).

### 11d) 4-(2,4-Difluoro-benzyl)-piperidine-1-carboxylic Acid 4-benzyloxy-benzoylamide

The title compound is prepared from 4-benzyloxy-benzoyl chloride and 4-(2,4-difluoro-benzyl)-piperidine according to the method described in Example 1a.

### 11e) 4-(2,4-Difluoro-benzyl)-piperidine-1-carboxylic Acid 4-hydroxy-benzoylamide

The title compound is prepared from 4-(2,4-difluoro-benzyl)-piperidine-1-carboxylic acid 4-benzyloxy-benzoylamide according to the method described in Example 4b. Mp.: 168° C.

### Example 12

### 4-Benzyl-piperidine-1-carboxylic Acid 4-methanesulfonylamino-benzoylamide

The title compound is prepared from 4-methanesulfonylamino-benzamide and 4-benzyl-piperidine according to the method described in Example 6. Mp.: 225-228° C.

The title compound is prepared from 1H-benzotriazol-5-carboxylic acid amide and 4-benzylpiperidine according to the method described in Example 18. Mp.: 97.5-100° C.

### Example 20

### 4-(4-Fluorobenzyl)piperidine-1-carboxylic acid (1H-benzotriazol-5-carbonyl)-amide [the other Tautomeric Form of the Compound is 4-4-fluorobenzyl-piperidin-1-carboxylic Acid (3H-benzotriazol-5-carbonyl)-amide]

The title compound is prepared from 1H-benzotriazol-5-carboxylic acid amide and 4-(4-fluorobenzyl)piperidine [J. Med. Chem., 35, 4903, (1992)] according to the method described in Example 18. Mp.: 125-129° C.

### Example 21

### 4-Benzylpiperidine-1-carboxylic Acid (1H-indol-5-carbonyl)-amide

The title compound is prepared from 1H-indol-5-carboxylic acid amide [Heterocycles, 34, 1169, (1992)] and 4-benzylpiperidine according to the method described in Example 18. Mp.: 110-112° C.

### Example 22

### 4-(4-Fluoro-benzyl)-piperidine-1-carboxylic Acid 4-acetylamino-benzoylamide

A mixture of 1.4 g (8 mmol) of 4-acetylamino-benzamide [J. Amer. Chem. Soc, 34, 694. (1912)], 1.05 ml (12 mmol) of oxalyl chloride and 8 ml of 1,2-dichloroethane is refluxed for 3 hours and then cooled to 5° C. A solution of 2.8 g (12 mmol) of 4-(4-fluoro-benzyl)-piperidine hydrochloride and 2.5 ml (18 mmol) of triethylamine in 8 ml of 1,2-dichloroethane is added drop wise below 10° C., and the reaction mixture is stirred at room temperature for 10 hours. Then 25 ml of water is added to the mixture, the organic layer is separated and the water phase is extracted three times with 20 ml of chloroform. The combined organic layers are dried over sodium sulfate, concentrated and the residue is purified by column chromatography using Kieselgel 60 as adsorbent (Merck) and chloroform:methanol=99:1 as eluent to yield 0.65 g (20%) of the title compound. Mp.: 156-171° C. (decomp. diethylether).

### Example 23

### 4-(4-Chloro-phenoxy)-piperidine-1-carboxylic Acid 4-acetylamino-benzoylamide

The title compound is prepared from 4-(4-chloro-phenoxy)-piperidine and 4-acetyl amino-benzamide according to the method described in Example 22. Mp.: 79° C. (decomp. diethylether).

### Example 24

### 4-(4-Fluoro-benzyl)-piperidine-1-carboxylic Acid 4-methanesulfonylamino Benzoylamide

The title compound is prepared from 4-(4-fluoro-benzyl)-piperidine and 4-methanesulfonylamino-benzamide according to the method described in Example 22. Mp.: 221-222° C. (ethanol).

### Example 25

### 4-(4-Chloro-phenoxy)-piperidine-1-carboxylic Acid 4-methanesulfonylamino Benzoylamide

The title compound is prepared from 4-(4-chloro-phenoxy)-piperidine and 4-acetyl amino-benzamide according to the method described in Example 22. Mp.: 79° C. (decomp. diethylether).

### Method B. (Solid Phase Synthesis)

### Example 26

### 4-(3-Methoxy-benzyl)-piperidine-1-carboxylic Acid 4-hydroxy-benzoylamide

### 26a) (3-Methoxy-benzyl)-piperidine

The title compound is prepared from N-(tert-butoxycarbonyl)-4-piperidone and (3-methoxy-benzyl)-phosphoric acid diethyl ester [J. Amer. Chem. Soc, 98, 5574-5581. (1976)] according to the method described in Example 11a-11c.

### 26b) 4-Hydroxybenzamide Anchored onto Resin

A mixture of 7.86 g (6.288 mmol) of Wang resin (Novabiochem; capacity: 0.8 mM/g; size: 100-200 mesh), 200 ml of tetrahydrofuran, 2.9 g (21.1 mmol) of 4-hydroxybenzamide (Aldrich), 6.3 g (24.0 mmol) of triphenylphosphine is stirred at 0° C. for 20 min, then 3.8 ml (241 mmol) of diethyl azodicarboxylate is added. The reaction mixture is stirred at 20° C. for 24 h, then the product is filtered off, washed twice with 300 ml of dimethylformamide, twice with 200 ml of tetrahydrofuran, twice with 300 ml of methanol and twice with 200 ml of tetrahydrofuran. The product is dried at room temperature to yield 8.8 g of the title compound. 26c) 4-(3-Methoxy-benzyl)-piperidine-1-carboxylic acid 4-hydroxy-benzoylamide anchored onto resin

To a mixture of 0.2 g (0.14 mmol) of 4-hydroxybenzamide obtained in the previous step in 4 ml of 1,2-dichloroethane 40 μl (0.46 mmol) of oxalyl chloride is added. The reaction mixture is shaken at 75° C. for 0.5 h, cooled to 20° C. and 150 μl (0.86 mmol) of N,N-diisopropylethylamine, 2 ml of 1,2-dichloroethane, 85 mg (0.41 mmol) of (3-methoxy-benzyl)-piperidine are added. The reaction mixture is shaken for 1 h. Then the resin is filtered off and washed five times with 4 ml of dichloromethane and three times with 4 ml of methanol, finally again twice with 4 ml of dichloromethane.

### 26d) 4-(3-Methoxy-benzyl)-piperidine-1-carboxylic Acid 4-hydroxy-benzoylamide

A mixture of 4-(3-methoxy-benzyl)-piperidine-1-carboxylic acid 4-hydroxy-benzoylamide anchored onto resin and 3 ml of a 1:10 mixture of trifluoroacetic acid:dichloromethane is shaken for 2 h. Then the resin is filtered off and washed twice with 1.5 ml of dichloromethane. The combined filtrate is concentrated. The residue is purified by column chromatography using Kieselgel 60 as adsorbent (Merck) and toluene:methanol=4:1 as eluent to yield 1.4 mg of the title compound. k′=4.163.

### Example 27

### 4-[2-(p-Tolyl)-ethyl]-piperidine-1-carboxylic acid 4-hydroxy-benzoylamide

The title, compound is prepared from 4-(2-p-tolyl-ethyl)-piperidine [Chem. Ber., 38, 161. (1905)] according to the method described in Example 26. k′=4.631.

### Example 28

### 4-(Phenylthio-methyl)-piperidine-1-carboxylic Acid 4-hydroxy-benzoylamide

### 28a). 4-(Phenylthio-methyl)-piperidine-1-carboxylic Acid tert-butyl Ester

Under argon, to a stirred solution of 1.1 ml (10.7 mmol) of benzenethiol (Aldrich) in 20 ml of dimethylformamide 0.5 g (60% , 12.5 mmol) of sodium hydride is added . The reaction mixture is stirred at 20° C. for 0.5 h, then 3.0 g (10.2 mmol) of 4-methanesulfonyloxymethyl-piperidine-1-carboxylic acid tert-butyl ester [Bioorg. Med. Chem. Lett., 11j 3161-3164. (2001)] in 10 ml of dimethylformamide is added drop wise at 20° C. The reaction mixture is stirred at 20° C. for 3 h, 1 ml of ethanol is added drop wise, poured into 100 ml of water and extracted with chloroform. The organic layer is dried over sodium sulfate and concentrated to yield 3.2 g of the title compound as oil.

### 28b) 4-(Phenylthio-methyl)-piperidine Hydrochloride

To a solution of 50 ml of 2.5 M hydrochloric acid in ethyl acetate 3.2 g (˜10 mmol) of 4-(phenylthio-methyl)-piperidine-1-carboxylic acid tert-butyl ester is added. The reaction mixture is stirred at 20° C. for 3 h. The precipitated crystals are filtered off, washed with ethyl acetate to yield 2.18 g (89%) of the title compound. Mp.: 183-184° C.

### 28d) 4-(Phenylthio-methyl)-piperidine-1-carboxylic Acid 4-hydroxy-benzoylamide

### Example 34

### 4-[2-(4-Methoxy-phenyl)-ethyl]-piperidine-1-carboxylic Acid 4-hydroxy-benzoylamide

The title compound is prepared from 4-[2-(4-methoxy-phenyl)-ethyl]-piperidine according to the method described in Example 26. k′=4.398.

### Example 35

### 4-(3-Cyano-benzyl)-piperidine-1-carboxylic Acid 4-hydroxy-benzoylamide

### 35a) 4-(3-Cyano-benzyl)-piperidine

The title compound is prepared from N-(tert-butoxycarbonyl)-4-piperidone and (3-cyano-benzyl)-phosphoric acid diethyl ester [Eur. J. Med. Chem., 15, 2927-2938. (2001)] according to the method described in Example 11a-11c.

### 35b) 4-(3-Cyano-benzyl)-piperidine-1-carboxylic Acid 4-hydroxy-benzoylamide

The title compound is prepared from 4-(3-cyano-benzyl)-piperidine according to the method described in Example 26. k′=4.048.

### Example 36

### 4-(2-Ethoxy-phenoxy)-piperidine-1-carboxylic Acid 4-hydroxy-benzoylamide

### 36a) 4-(2-Ethoxy-phenoxy)-piperidine

The title compound is prepared from 1-ethoxy-2-fluoro-benzene [Chem. Zentralbl., 84, 760. (1913)] according to the method described in Example 9a-9b.

### 36b) 4-(2-Ethoxy-phenoxy)-piperidine-1-carboxylic acid 4-hydroxy-benzoylamide

The title compound is prepared from 4-(2-ethoxy-phenoxy)-piperidine according to the method described in Example 26. k′=3.956.

### Example 37

### 4-(3-Fluoro-benzyl)-piperidine-1-carboxylic Acid 4-hydroxy-benzoylamide

### 37a) 4-(3-Fluoro-benzyl)-piperidine

The title compound is prepared from N-(tert-butoxycarbonyl)-4-piperidone and (3-fluoro-benzyl)-phosphoric acid diethyl ester [Org. Magn. Reson., 9, 35 (1977)] according to the method described in Example 11a-11c.

37b) 4-(3-Fluoro-benzyl)-piperidine-1-carboxylic Acid 4-hydroxy-benzoylamide

The title compound is prepared from 4-(3-fluoro-benzyl)-piperidine according to the method described in Example 26. k′=4.256.

### Example 38

### 4-Phenoxy-piperidine-1-carboxylic Acid 4-hydroxy-benzoylamide

The title compound is prepared from 4-phenoxy-piperidine [J. Med. Chem., 17, 1000. (1974)] according to the method described in Example 26. k′=3.786.

### Example 39

### 4-[1-(4-Hydroxy-benzoylcarbamoyl)-piperidine-4-yl-methyl]-benzoic Acid Methyl Ester

### 39a) 4-(4-Methoxycarbonyl-benzyl-piperidine

The title compound is prepared from N-(tert-butoxycarbonyl)-4-piperidone and (4-methoxycarbonyl-benzyl)-phosphoric acid diethyl ester [DE 1112072] according to the method described in Example 11a-11c.

### 39b) 4-[1-(4-Hydroxy-benzoylcarbamoyl)-piperidine-4-yl-methyl]-benzoic Acid Methyl Ester

The title compound is prepared from 4-(4-methoxycarbonyl-benzyl)-piperidine according to the method described in Example 26. k′=3.935.

### Example 40

### Preparation of Pharmaceutical Compositions

### a) Tablets:

0.01-50% of active ingredient of formula (L) 15-50% of lactose, 15-50% of potato starch, 5-15% of polyvinyl pyrrolidone, 1-5% of talc, 0.01-3% of magnesium stearate, 1-3% of colloid silicon dioxide and 2-7% of ultraamylopectin are mixed, then are granulated by wet granulation and pressed to tablets.

b) Dragées, Filmcoated Tablets:

The tablets made according to the method described above are coated by a layer consisting of entero- or gastrosolvent film, or of sugar and talc. The dragées are polished by a mixture of beeswax and carnuba wax.

### c) Capsules:

0.01-50% of active ingredient of formula (I), 1-5% of sodium lauryl sulfate, 15-50% of starch, 15-50% of lactose, 1-3% of colloid silicon dioxide and 0.01-3% of magnesium stearate are thoroughly mixed, the mixture is passed through a sieve and filled in hard gelatin capsules.

### d) Suspensions:

Ingredients: 0.01-15% of active ingredient of formula (I), 0.1-2% of sodium hydroxide, 0.1-3% of citric acid, 0.05-0.2% of nipagin (sodium methyl 4-hydroxybenzoate), 0.005-0.02% of nipasol, 0.01-0.5% of carbopol (polyacrylic acid), 0.1-5% of 96% ethanol, 0.1-1% of flavoring agent, 20-70% of sorbitol (70% aqueous solution) and 30-50% of distilled water.

To solution of nipagin and citric acid in 20 ml of distilled water, carbopol is added in small portions under vigorous stirring, and the solution is left to stand for 10-12 h. Then the sodium hydroxide in 1 ml of distilled water, the aqueous solution of sorbitol and finally the ethanolic raspberry flavor are added with stirring. To this carrier the active ingredient is added in small portions and suspended with an immersing homogenizator. Finally the suspension is filled up to the desired final volume with distilled water and the suspension syrup is passed through a colloid milling equipment.

### e) Suppositories:

For each suppository 0.01-15% of active ingredient of formula (I) and 1-20% of lactose are thoroughly mixed, then 50-95% of adeps pro suppository (for example Witepsol 4) is melted, cooled to 35° C. and the mixture, of active ingredient and lactose is mixed in it with homogenizator. The obtained mixture is mould in cooled forms.

### f) Lyophilized Powder Ampoule Compositions:

A 5% solution of mannitol or lactose is made with bidistilled water for injection use, and

the solution is filtered so as to have sterile solution. A 0.01-5% solution of the active ingredient of formula (I) is also made with bidistilled water for injection use, and this solution is filtered so as to have sterile solution. These two solutions are mixed under aseptic conditions, filled in 1 ml portions into ampoules, the content of the ampoules is lyophilized, and the ampoules are sealed under nitrogen. The contents of the ampoules are dissolved in sterile water or 0.9% (physiological) sterile aqueous sodium chloride solution before administration.

What we claim is:

1. New benzoyl urea derivatives of formula (I) 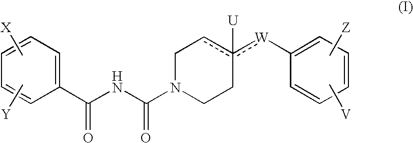
wherein the meaning of
X and Y independently are hydrogen atom, hydroxy, benzyloxy, amino, nitro, C₁-C₄ alkylsulfonamido optionally substituted with a halogen atom or halogen atoms, C₁-C₄ alkanoylamido optionally substituted with a halogen atom or halogen atoms, C₁-C₄ alkoxy, aroyl-carbamoyl optionally substituted with halogen atom or C₁-C₄ alkyl or C₁-C₄ alkoxycarbonyl group, or
the neighboring X and Y groups optionally form together with one or more identical or different additional hetero atom and —CH═ and/or —CH₂— groups an optionally substituted 4-7 membered homo- or heterocyclic ring, preferably morpholine, pyrrole, pyrrolidine, oxo- or thioxo-pyrrolidine, pyrazole, pyrazolidine, imidazole, imidazolidine, oxo- or thioxo-imidazole or imidazolidine, 1,4-oxazine, oxazole, oxazolidine, triazole, oxo- or thioxo-oxazolidine, or 3-oxo-1,4-oxazine ring,
V and Z independently are hydrogen or halogen atom, cyano, C₁-C₄ alkyl, C₁-C₄ alkoxy, trifluoromethyl, hydroxy or optionally esterized carboxyl group,
W is oxygen atom, as well as C₁-C₄ alkylene, C₂-C₄ alkenylene, aminocarbonyl, —NH—, —N(alkyl)-, —CH₂O—, —CH₂S—, —CH(OH)—, —OCH₂— group, —wherein the meaning of alkyl is a C₁-C₄ alkyl group—,
when the dotted bonds [symbol] represent simple C—C bonds then U is hydroxy group, or hydrogen atom or
when W is C₁-C₄ alkylene or C₂-C₄ alkenylene group, then one of the dotted bonds [symbol] can represent a further double C—C bond and in this case U means ah electron pair, which participate in the double bond
and optical antipodes, racemates and the salts thereof.

2. Compounds of formula (I) as defined in claim 1, wherein the meaning of
X is hydrogen atom,
Y is hydroxy, benzyloxy-, amino, nitro, C₁-C₄ alkylsulfonamido, C₁-C₄ alkanoylamido, benzoyl-carbamoyl optionally substituted with halogen atom or C₁-C₄ alkyl, C₁-C₄ alkoxycarbonyl group, or
the neighboring X and Y groups optionally form together with one or more identical or different additional hetero atom and —CH═ and/or —CH₂— groups an oxazole, imidazole or triazole ring,
V and Z independently are hydrogen or halogen atom, cyano, C₁-C₄ alkyl, C₁-C₄ alkoxy, trifluoromethyl, hydroxy or methoxy-carbonyl group,
W is oxygen atom, as well as C₁-C₄ alkylene, —CH₂O—, —OCH₂— group,
when the dotted bonds [symbol] represent simple C—C bonds then the meaning of U is hydroxy group or hydrogen atom or
when W is C₁-C₄ alkylene or C₂-C₄ alkynylene group, then one of the dotted bonds [symbol] can represent a further double C—C bond and in this case U means an electron pair, which participate in the double bond.

3. A compound of the following group of benzoyl urea derivatives belonging to the scope of claim 1
4-benzyl-piperidine-1-carboxylic acid 4-hydroxy-benzoylamide,
4(4-methoxy-benzyl)-piperidine-1-carboxylic acid 4-hydroxy-benzoylamide,
4-(4-methyl-benzyl)-piperidine-1-carboxylic acid 4-hydroxy-benzoylamide,
4-(4-chloro-benzyl)-piperidine-1-carboxylic acid 4-hydroxy-benzoylamide,
4-(4-fluoro-benzyl)-piperidine-1-carboxylic acid 4-hydroxy-benzoylamide,
4-(4-methyl-benzyl)-piperidine-1-carboxylic acid 4-methanesulfonylamino benzoylamide,
4-benzyl-piperidine-1-carboxylic acid (2-oxo-2,3-dihydro-benzooxazole-6-carbonyl)-amide,
4-(3-methoxy-benzyl)-piperidine-1-carboxylic acid 4-hydroxy-benzoylamide,
4-(2-p-tolyl-ethyl)-piperidine-1-carboxylic acid 4-hydroxy-benzoylamide,
4-(phenylthio-methyl)-piperidine-1-carboxylic acid 4-hydroxy-benzoylamide,
4-(4-trifluoromethyl-benzyl)-piperidine-1-carboxylic acid-4-hydroxy-benzoylamide.

4. Pharmaceutical compositions containing an effective amount of the benzoyl urea derivatives of formula (I)—wherein the meaning of X, Y, V, W, Z, the dotted bonds [symbol] and U are as given in claim 1—or optical antipodes or racemates or the salts thereof as active ingredients and auxiliary materials, which are commonly used in practice, such as carriers, excipients, diluents, stabilizers, wetting or emulsifying agents, pH—and osmotic pressure-influencing, flavoring or aromatizing, as well as formulation-promoting or formulation-providing additives.

5. Process for preparing the benzoyl urea derivatives of formula (I) 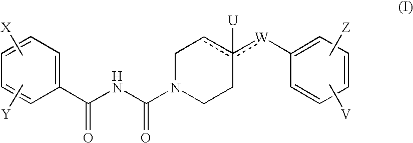
wherein the meaning of X, Y, V, W, Z, the dotted bonds [symbol] and U are as given in claim 1—, characterized by
a.) reacting a substituted benzoyl isocyanate of formula (II) preferably synthesized in situ 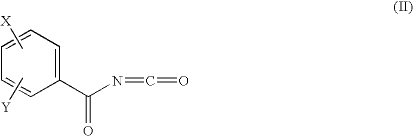
wherein the meaning of X and Y are as given in claim 1—with an amine of formula (III) 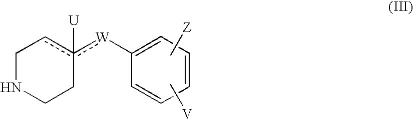
wherein the meaning of V, W, Z, the dotted bonds [symbol] and U re as given in claim 1—, or
b.) coupling a substituted benzamide of formula (V) 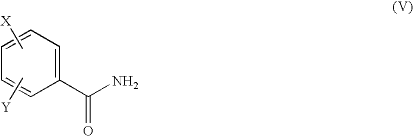
where X is hydroxy and Y is as given in claim 1—onto a resin using triphenyl phosphine and diethyl azodicarboxylate, then
reacting the obtained benzamide coupled to resin with oxalyl chloride and the so formed benzoyl isocyanate with an amine of formula (III) 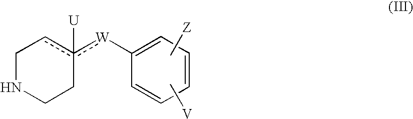
wherein the meaning of V, W, Z, the dotted bonds [symbol] and U are as given in claim 1—in the presence of a trialkyl amine,
and splitting off the obtained benzoyl urea derivatives of formula (I)—wherein the meaning of X, Y, V, W, Z, the dotted bonds [symbol] and U are as given in claim 1—from the resin,
then optionally transforming the so obtained benzoyl urea derivatives of formula (I)—wherein the meaning of X, Y, V, W, Z, the dotted bonds [symbol] and U are as given in claim 1—, into another benzoyl urea derivatives of formula (I) by introducing new substituents and/or modifying or removing the existing ones, and/or by salt formation and/or by liberating the compound from salts, and/or by resolving the obtained racemates using optically, active acids or bases by known methods.

6. Process as claimed in claim 5, characterized by starting from a substituted benzoyl isocyanate of formula (II)—wherein the meaning of X and Y are as given in claim 1—synthesized by reacting a substituted benzoyl halogenide of formula (IV) 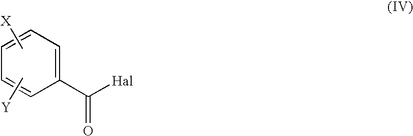
where X and Y are as given in claim 1 and Hal is a halogen atom—with an alkali metal cyanate in presence of tin(IV) chloride.

7. Process as claimed in claim 5, characterized by starting from a substituted benzoyl isocyanate of formula (II)—wherein the meaning of X and Y are as given in claim 1—synthesized by reacting a substituted benzamide of formula (V) 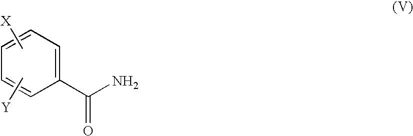
where X and Y are as given in claim 1—with oxalyl chloride.

8. Process for manufacturing pharmaceutical compositions having NR2B selective NMDA receptor antagonist effect, characterized by mixing an effective amount of the benzoyl urea derivatives of formula (I)—wherein the meaning of X, Y, V, W, Z, the dotted bonds [symbol] and U are as given in claim 1—or optical antipodes or racemates or the pharmaceutically acceptable salts thereof as active ingredients and auxiliary materials, which are commonly used in practice, such as carriers, excipients, diluents, stabilizers, wetting or emulsifying agents, pH—and osmotic pressure-influencing, flavoring or aromatizing, as well as formulation-promoting or formulation-providing additives.

9. Method of treatment and alleviation of symptoms of the following diseases of mammals—including human—traumatic injury of brain or spinal cord, human immunodeficiency virus (HIV) related neuronal injury, amyotrophic lateral sclerosis, tolerance and/or dependence to opioid treatment of pain, withdrawal syndromes of e.g. alcohol, opioids or cocaine, ischemic CNS disorders, chronic neurodegenerative disorders, such as Alzheimer's disease, Parkinson's disease, Huntington's disease, pain and chronic pain states, such as neuropathic pain or cancer related pain, epilepsy, anxiety, depression, migraine, psychosis, muscular spasm, dementia of various origin, hypoglycemia, degenerative disorders of the retina, glaucoma, asthma, tinnitus, aminoglycoside antibiotic-induced hearing loss, characterized by administering effective amount/amounts of benzoyl urea derivatives of formula (I)—wherein the meaning of X, Y, V, W, Z, the dotted bonds [symbol] and U are as given in claim 1—or optical antipodes or racemates or the pharmaceutically acceptable salts thereof as such or combined with carriers, filling materials and the like usually applied in pharmaceuticals to the mammal to be treated.

10. Use of benzoyl urea derivatives of formula (I)—wherein the meaning of X, Y, V, W, Z, the dotted bonds [symbol] and U are as given in claim 1—and/or optical antipodes or racemates and/or pharmaceutically acceptable salts thereof for the preparation of a pharmaceutical for the treatment and alleviation of symptoms of the following diseases in a mammals, including humans: traumatic injury of brain or spinal cord, human immunodeficiency virus (HIV) related neuronal injury, amyotrophic lateral sclerosis, tolerance and/or dependence to opioid treatment of pain, withdrawal syndromes of e.g. alcohol, opioids or cocaine, ischemic CNS disorders, chronic neurodegenerative disorders, such as Alzheimer's disease, Parkinson's disease, Huntington's disease, pain and chronic pain states, such as neuropathic pain or cancer related pain, epilepsy, anxiety, depression, migraine, psychosis, muscular spasm, dementia of various origin, hypoglycemia, degenerative disorders of the retina, glaucoma, asthma, tinnitus, aminoglycoside antibiotic-induced hearing loss.
